(19) United States
(12) Patent Application Publication
(10) Pub. No.: US 2010/0268156 A1
(43) Pub. Date: Oct. 21, 2010

(54) MEDICAL DEVICE IN THE FORM OF A CATHETER FOR SUPPLYING AND/OR REMOVING FLUID INTO AND/OR FROM, ESPECIALLY BODY CAVITIES, ESPECIALLY THE PLEURAL CAVITY

(76) Inventors: Walter Milacek, Wein (AT); Christian Sabeff, Wien (AT)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 12/741,441

(22) PCT Filed: Oct. 28, 2008
(86) PCT No.: PCT/AT08/00391

§ 371 Date: May 5, 2010

(30) Foreign Application Priority Data
Nov. 6, 2007 (AT) A 1784/2007
Mar. 5, 2008 (AT) A 361/2008

(51) Int. Cl. A61M 1/00; A61M 5/32; A61M 25/14

(52) U.S. Cl. 604/30; 604/247; 604/319

(57) ABSTRACT

The invention relates to a medical device that is in the form of a catheter (3) for supplying fluid to but in particular removing fluid from body cavities. The catheter head (1) on the proximal end (4) comprises an inlet (5) that is provided with one or more sealing and/or blocking elements (6, 7) and that is used to introduce a hollow or Verres needle (18) into the catheter (3). In the region between the sealing or blocking element(s) and the region of the catheter shaft (2) that is introduced into the body, a branch connection (8 ) is arranged, said branch connection being connected to a suction pump arrangement (10) via a branch tube (9 ). Said branch tube (9) leads to a suction valve (11) that enables a fluid to flow in the direction of the suction device, but blocks the flow in the counter direction. The outlet of the suction valve (11) opens into a forked area (12), from which one branch (16) leads to a suction device connection, in particular an injection connection (13) that is embodied, preferably, as a Luer-lock, and the other branch thereof (17) leads to a secretion bag connection (14). A non-return valve (15) that blocks the return flow of the secretion bag is arranged between said branch (17) and the secretion bag connection (14).

The present invention concerns a medical device in the form of a catheter for supplying and/or removing fluid into and/or from, especially body cavities, especially the pleural cavity, the device exhibiting a catheter head and a catheter shaft, where the catheter head exhibits at the proximal end an inlet provided with one or several sealing and/or stop valves for inserting a hollow or Verres needle into the catheter, and where in the area between the sealing and/or stop valve(s) and the section of the catheter shaft designed for insertion into the body there is provided a branch connection, which is connected preferably via a branching hose to a suction pump arrangement.

The device according to the invention can be used to remove air or bodily fluids via a puncture from the human body, however it can find application in the same manner in the veterinary field, where the present description discusses especially the use in human medicine. preferred application possibility arises in the treatment of pathological fluid or gas build-ups in the thoracic space (pleural cavity).

The lung tissue is surrounded by the visceral pleura like a cloak. At the same time the costal pleura clothes the inner side of the ribs. The pleura cavity is described as The space between the visceral and the costal pleura is referred to as the pleural cavity, which in healthy humans is empty of air and fluids, where normally low pressure prevails in this gap.

Pleural effusions arise when pleural fluid is produced excessively, or if lymphatic drainage is reduced, with the possible causes being manifold. One common cause is malignant tumours, but injuries, inflammations or cardiac insufficiency can also lead to pleural effusions. Even fluid build-ups of 20 to 30 millilitres can be detected by means of an ultrasound examination, but because pleural effusions arise gradually and the symptoms mostly occur only late, by the time of the diagnosis often there are already several litres of fluid in the pleural cavity, where often to begin with it is unclear what fluid the effusion consists of (bloody, purulent, clear, etc.). Clarity about this can be achieved through a diagnostic pleural puncture, where the obtained fluid can be examined chemically, cytologically and bacteriologically.

The treatment for pleural effusions depends fundamentally on the underlying illness, where in the case of large effusion quantities a relief puncture can be sensible in order to reduce the amount of accumulated fluid. In order to avoid cardiovascular stress and in order to reduce the risk of a pulmonary oedema, in general not the entire fluid quantity is removed at one time, but mostly between 100 and a maximum of 1500 ml. In order to prevent inflammations or in order to be able to treat them, medications, for example antibiotics, can also be introduced into the pleural cavity via the puncture site. In the case of purulent effusion in the pleural cavity (pleural empyema), irrigation-suction drainage may be necessary in the pleural cavity, in which consolidated materials in the pleural cavity are loosened by introducing an irrigation fluid before and between the suction steps.

The pleural puncture is mostly performed by an experienced physician with the patient under local anaesthetic, where a needle (or a puncture catheter) is introduced into the space between the costal pleura and visceral pleura (pleural gap), under ultrasound inspection if necessary. The puncture can on the one hand serve for obtaining material for examination (diagnostic application), and on the other the fluid accumulations can be suctioned off (therapeutic application). Mostly a cannula or a hollow needle encased in a synthetic tube is used, which is introduced between two ribs through the skin and the intercostal musculature into the pleural cavity. Through a syringe attached on the cannula, a little pleural effusion can be suctioned off and taken to a laboratory for further examination. Afterwards the hollow needle is pulled out and capillary hose connected to the remaining synthetic tube, through which the effusion is drained. The connecting elements used are mostly those known as Luer connectors or Luer locks, since they are used internationally and make possible a uniform standardised (e.g. in DIN 13090, EN 1707 or ISO 594/1) linking of various medical devices.

The suctioning off can take place via a syringe, or through special low-pressure or pump systems that are attached to the catheter and serve especially for draining large quantities of liquids or gases. For pumping away liquids, usually a simple suction device is used, consisting essentially of an adjustable three-way valve, with to one branch of the valve is attached a syringe, mostly via a Luer lock, the other branch leads to a collecting bag for the suctioned-off secretion, and the third branch making the connection to the pleural cavity via the pleural puncture catheter. For suctioning, first the valve is so adjusted with a manual lever that the connection to the collecting bag is cut off, and the fluid suctioned off from the pleural cavity into the syringe by drawing up the syringe. Once the syringe is filled, the valve lever is changed over such that the connection to the catheter is closed, and the suctioned-off liquid is now pushed, by compressing the syringe, into the collecting bag, which is now connected via the valve with the syringe. This arrangement requires constant manual changing-over of the valve, where a mistake in changing-over could lead to the fluid being suctioned out of the collecting bag and/or even injected back into the pleural cavity.

After the suctioning-off procedure, medications can be introduced into the pleural cavity via the catheter if necessary, for example a local anaesthetic, an antibiotic, tissue adhesive, a cell-killing agent (cytostatic) or painkillers. Finally the catheter's synthetic tube is withdrawn and a bandage applied.

In conventional catheters, often another device must be attached to the catheter between individual working steps, where it must always be ensured that no air can penetrate the pleural cavity as this could quickly lead to a pneumothorax (gas in the chest). Various stop valves serve for this, whose correct operation is the physician's responsibility. In addition to operating the devices, the physician also has to give instructions to the patient, for example when to hold his breath or to exhale or inhale. Thus errors are avoided solely through the physician's technical competence and vigilance, as the devices used do not prevent faulty operation.

The possible complications that can occur when placing a pleural catheter include infections, but this is rare with a sterile needle and good skin disinfection. In addition it is possible for the lung to be injured when introducing the catheter or if the patient moves or coughs. Injury to the liver or spleen and secondary bleeding may occur also.

In order to prevent injury to the lung and to the internal organs, it is for example possible to use for the puncture special needles referred to as “Verres needles” , which have been known in this technical field for a long time. Verres needles permit the piercing of hard material (muscle tissue, costal pleura), but prevent injury to soft material (visceral pleura, lung tissue). For this purpose, a Verres needle consists of an outer needle with a sharp distal end (similar to a cannula), in whose lumen there extends an inner probe with a blunt probe tip. The inner probe is pre-tensioned forward by means of a spring, such that its blunt probe tip extends past the tip of the outer needle. When piercing relatively solid tissue, such as e.g. the chest musculature, the probe tip is pushed back into the outer needle against the force of the spring, such that the sharp end of the outer needle is exposed and can penetrate the tissue like a normal cannula tip. As soon as the tip of the needle reaches softer tissue, the probe tip pushes itself again out of the outer needle through the force of the spring and thereby pushes the softer tissue away from the tip of the outer needle, such that the tissue is not damaged. To be able to use the Verres needle like a conventional cannula, the probe is designed to be hollow, with the distal opening being provided not at the tip but laterally from it.

The use of a Verres needle in a medical instrument for a pleural puncture is described for example in DE 693 28 254. DE 693 28 254 also discloses a ball valve, which as soon as the Verres needle is withdrawn from the catheter, closes the needle inlet of the pleural puncture catheter in order to prevent the ingress of air into the pleural cavity, with the valve consisting of several movable parts (spring, ball, piston). The head of the catheter exhibits a branching, from which a hose leads to a suction unit which in conventional fashion uses a three-way valve, as described above.

In recent times, check valves made of an elastic material such as e.g. rubber or another elastomer, are being used increasingly in the medical field. These valves consist of a generally one-part body, which mostly exhibits an inner cavity, essentially of a cylindrical form, and tapers down in a wedge shape on one side, such that at the tip of the wedge the inner cavity space is closed off through two connected lip sealing lips abutting against each other. These valves are generally referred to as lip valves or due to their form as (duck-)bill valves. Due to their simple construction, these valves are available in many configuration, where the tip does not always have to be wedge-shaped but in some models can also be designed to be conically tapered or cross-shaped (similar to a Phillips screwdriver). Lip valves are cost-effective, fit into the smallest space, require only a simple housing, are corrosion and wear-resistant, self-cleaning and safe and easy to assemble. Lip valves can also be designed for the widest range of pressure differences and can be tailored to meet specific demands by varying their shape and material. Various models of lip valves for the medical field can be found, for example, in the product catalogues of Vernay Laboratories, Inc, Yellow Springs, Ohio, USA.

Furthermore, the use of a lip valve arranged in a branching of an endotracheal tube as a sealing entry port for a catheter is known from WO2006/103233.

It is an aim of the present invention to provide a pleural puncture catheter which reliably prevents the penetration of air into the pleural cavity, while being simpler in application and more cost-effective to manufacture than medical devices currently used for pleural puncture. In order to solve these tasks, a device according to the invention is being proposed whose branching hose leads to a suction valve that allows the flow of a liquid in the direction of the suction device but blocks the flow in the opposite direction, where the outlet of the suction valve opens into a bifurcation, from which one branch leads to a suction device connector preferably designed as a Luer lock, especially a syringe connector, and whose other branch leads to a secretion bag connector, where between this branch and the secretion bag connector there is arranged a check valve that blocks the backflow from the secretion bag.

The medical device designed according to the invention makes possible simple and safe puncture of the pleural cavity or another body cavity, while over the entire duration of treatment the penetration of air or foreign substances into the inside of the body is prevented. Furthermore, the medical device according to the invention contains a simple and effective suctioning-off system whose handling is less complex than in current systems, and which is suitable for suctioning off both liquids and gases. In particular, the physician does not need to change over valves manually during suctioning, such that he can direct his attention more on the patient's treatment instead of on operating the device. With the catheter according to the invention, the supply of fluid into the body cavity is possible also, without the catheter device or to parts thereof having to be replaced. This possibility can be used for example in order to inject medications into the pleural cavity after suctioning off, or in order to use the device as irrigation-suction drainage, where consolidated substances in the body cavity are loosened before suctioning through irrigation.

In one advantageous embodiment, the secretion bag connector can be designed as a Luer lock, where the check valve which blocks the backflow from the secretion bag can be a bill or lip valve or a membrane check valve, which is arranged on the inside of the Luer lock. The suction valve can also be designed as a bill or lip valve or a membrane check valve. Bill or lip valves (which are also known under the name “duckbill valve”) are especially simple and low-cost valves and are therefore especially suitable for use in disposable devices. Membrane check valves are also cost-effective to manufacture and moreover have the advantage of very small installation dimensions. Arranging the valve in the Luer lock of the secretion bag connector constitutes an especially advantageous embodiment, as the number of different functional units is minimised.

In a further embodiment, the branching hose between the catheter and the suction valve can preferably be sealable by means of a hose clamp. As a result, the connection from the catheter to the suction pump arrangement can be interrupted rapidly and in an uncomplicated fashion, such that catheter can be used for introducing fluid into the body.

A further embodiment of the invention envisages that the inlet for introducing the hollow or Verres needle can exhibit a bill or lip valve directed inwards, designed as a push-through valve, which abuts the circumference of the pushed-in hollow or Verres needle as a seal, and which with the needle withdrawn blocks the passage of fluid in both directions under the pressure differences that usually occur between the body cavity and the outside. In addition, a preferably manually operated stop valve can be arranged in the catheter head between the push-through valve and the branch connector. As a result, the needle that serves for the insertion of the catheter can be easily withdrawn from the catheter as soon as the latter has been brought into position. Through the valve, the needle inlet is sealed immediately such that no air can penetrate inside the body. A manually operated stop valve serves for additional reliability and seals the needle inlet even under higher pressure differences, which can occur for example during pumping-off or inhaling. If the stop valve is opened, liquids can be introduced into the body cavity via the catheter, for example with a syringe. To this end, the push-through valve does not necessarily have to be pierced with a needle, instead a syringe attached to the catheter head via a Luer connector suffices, as the valve opens under pressure differences that exceed the opening pressure and allows the introduction of liquids. This closure of the needle inlet is, moreover, technically simpler and considerably more cost-effective to manufacture than known systems that use ball valves for example.

In a further embodiment, the suction pump arrangement, including the suction valve, the bifurcation, the branch to the syringe connector, the branch to the secretion bag connector, the syringe connector, the secretion bag connector and the check valve can be integrated into one combined component. It is especially advantageous here if the suction pump arrangement is designed as a T-piece, where the suction valve and/or the check valve is designed as a membrane check valve. Such a T-piece can be manufactured with relatively few individual components and offers advantages during machine fabrication, since when manufacturing the T-piece by means of an injection moulding method simple injection moulds can be used that that can manage without a slider. Through the use of membrane check valves as suction and/or check valves, an especially compact design of the suction pump arrangement can be achieved.

Special embodiments of the invention are described below by reference to exemplifying drawings.

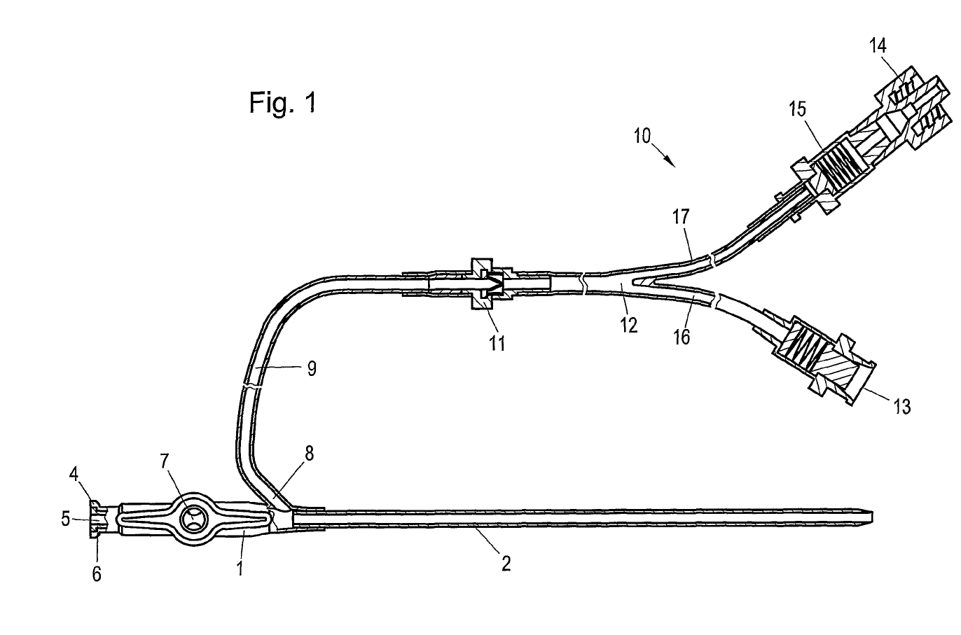

FIG. 1 shows a catheter according to the invention with the suction pump arrangement attached to it in a cutaway view,

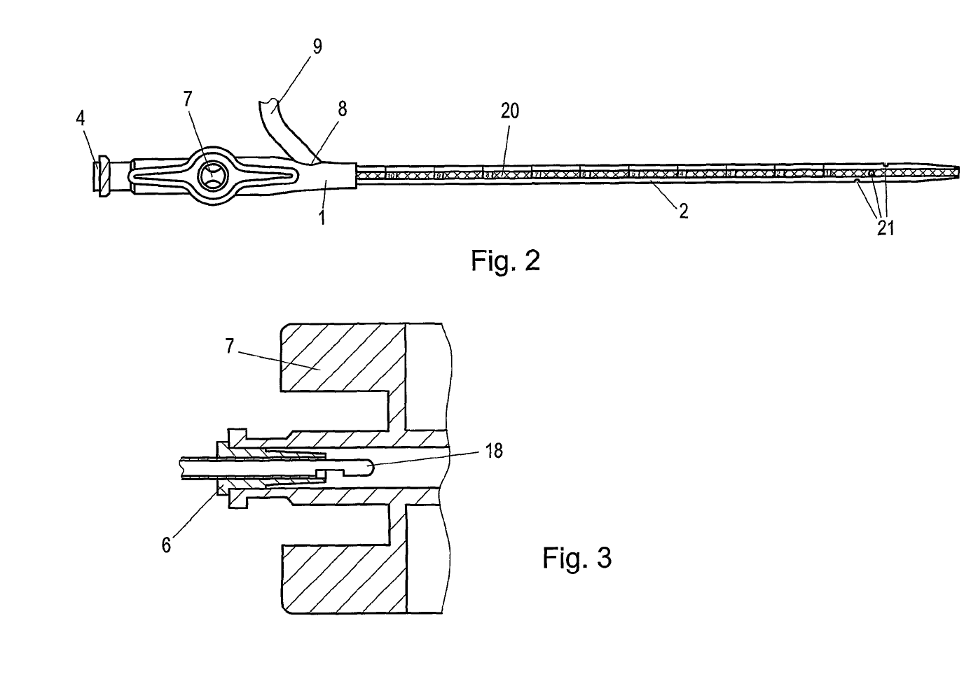

FIG. 2 shows the catheter without the suction pump arrangement in a plan view,

FIG. 3 shows the needle inlet of the catheter head with the tip of a Verres needle arranged inside it and the stop valve in a cutaway view,

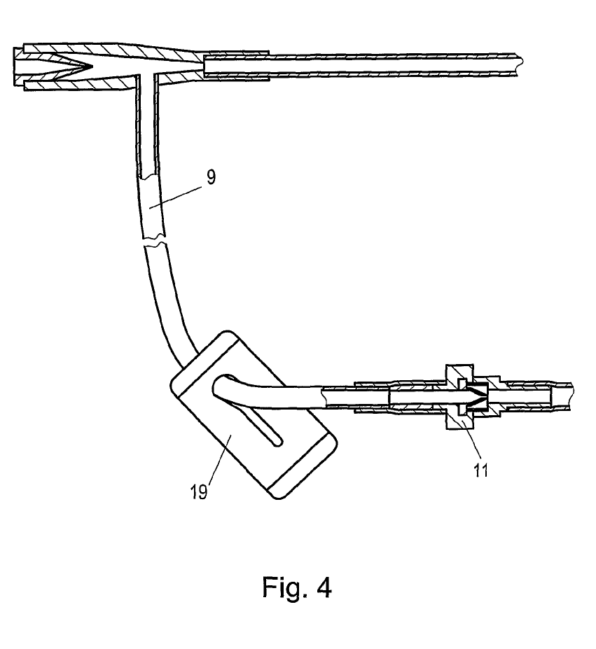

FIG. 4 shows a catheter according to the invention with a hose clamp provided at the branching hose,

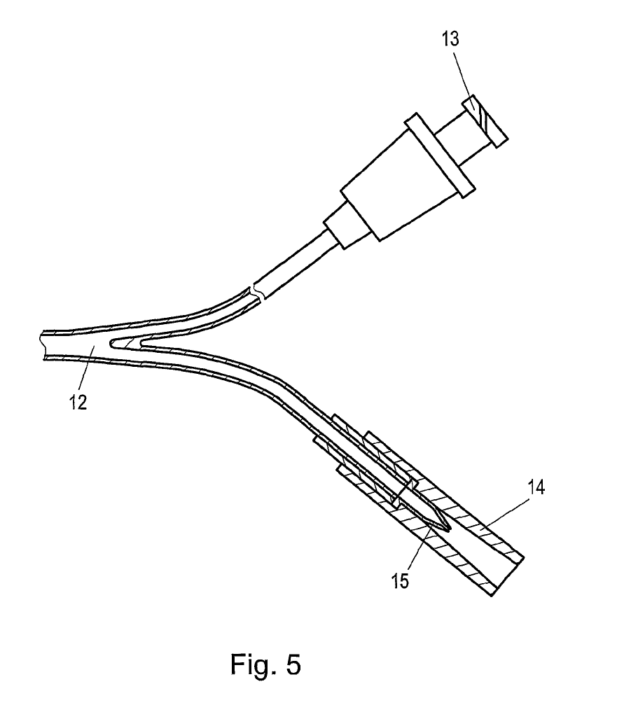

FIG. 5 shows a part of the suction pump arrangement according to the invention, where a check valve designed as a bill valve is arranged on the inside of the secretion bag connector,

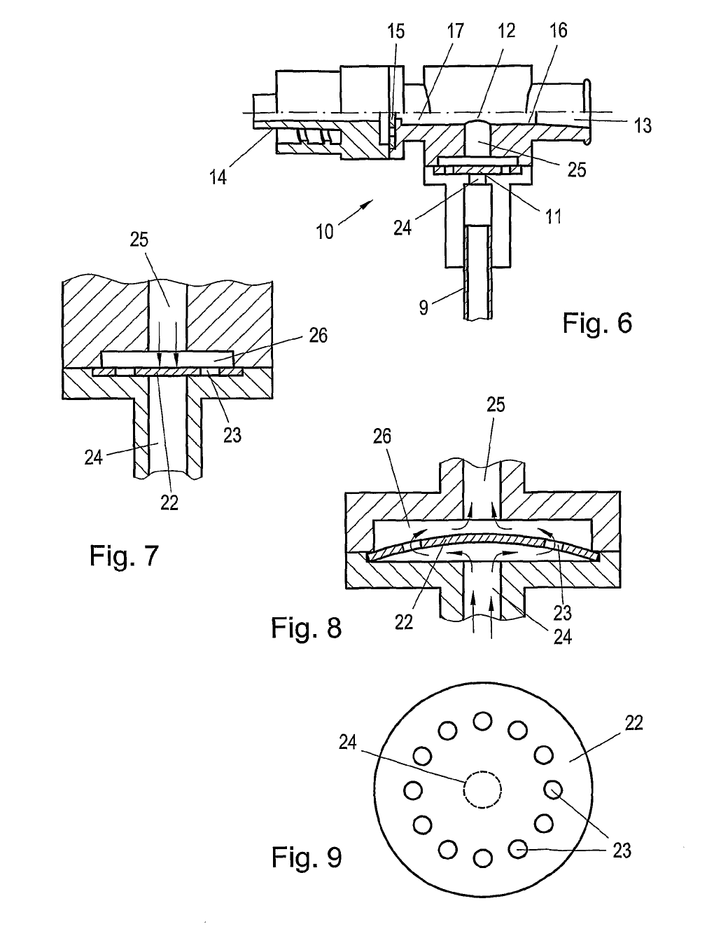

FIG. 6 shows a suction pump unit designed as a T-piece in a partly sectional view,

FIG. 7 shows the membrane check valve used as a suction valve in the intake valve T-piece of FIG. 6 in an enlarged view,

FIG. 8 shows in a schematic view the functional principle of the membrane check valve,

FIG. 9 shows a plan view of the valve membrane, and

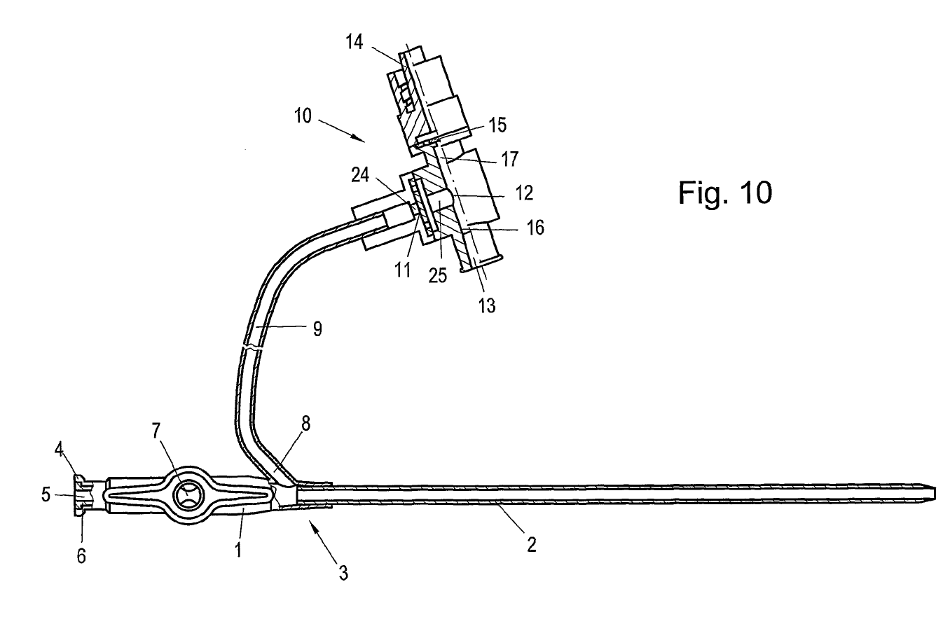

FIG. 10 shows the entire catheter arrangement with the T-piece.

FIG. 1 shows a pleural puncture catheter 3 according to the invention with the suction pump arrangement 10, which is connected with the catheter 3 via a branching hose 9, which leads away from a branch connector 8 of the catheter 3. The catheter 3 consists essentially of a catheter shaft 2 and a catheter head 1, where a stop valve 7 is connected at the catheter head, which in the open position allows the insertion of a needle into the catheter, and in the closed position closes off the catheter head. At the proximal end 4 of the catheter 3 there is arranged a push-through valve 6 as a needle inlet 5, which is represented in greater detail in a detailed section view in FIG. 3. FIG. 3 shows the tip of a Verres needle 18, that has just been pushed into the needle inlet 5. In doing so the push-through valve seals the needle at its perimeter. With the needle inserted fully into catheter 3, the catheter shaft 2 can be pushed in the familiar manner into the body cavity, especially the pleural cavity, with the tip of the needle protruding a little over the distal end of the catheter shaft 2. This procedure can be monitored by means of imaging methods, such as e.g. ultrasound or x-ray techniques, with the catheter shaft 2 exhibiting a strip 20 opaque to x-rays (FIG. 2), on which, for example every ten millimetres, markings are provided such that the dimensions can be identified on an x-ray. At the distal end of the catheter shaft 2 there are provided four outlets 21 arranged around the perimeter of the shaft in spiral fashion, which improve fluid communication with the body cavity.

Once the catheter 3 has been placed and if necessary its correct position has been checked, the needle is withdrawn from the catheter such that only the flexible catheter shaft 2 remains in the body of the patient. The push-through valve 6 arranged in inlet 5 exhibits preferably an opening pressure which is so high that the valve does not yet open under the pressure differences expected between the body cavity and the environment. Consequently with the needle withdrawn, the valve prevents air being sucked into the body. This is important especially during treatment of the pleural cavity, as a poorly sealing valve could lead to the penetration of air into the pleural cavity, and in the worst case to a tension pneumothorax, which constitutes a life-threatening complication. However an experienced physician is likely to identify this situation immediately and be able to stop it.

A stop valve 7, which is arranged between the needle inlet 5 and the branch connector 8 at the catheter head 1, serves additionally for further operational safety of the device. Preferably, the needle is retracted first to the position shown in FIG. 3, so that the stop valve 7 can be closed before the needle 18 is withdrawn completely from the needle inlet 5. With this procedure, valves with a lower opening pressure can be used also, without a potential risk to the patient through the penetration of air. Optionally, the needle inlet 5 could also be closed off with a simple cap as soon as the needle has been removed.

If the catheter has now been brought into position and the needle inlet 5 closed off, suction of the liquid or the gas from the pleural cavity can be started. The suction pump arrangement 10 serves this purpose, consisting essentially of a suction valve 11, a bifurcation 12, a syringe connector 13, a secretion bag connector 14 and the connecting hoses which lie in between. The suction pump arrangement 10 is linked to the catheter via a branching hose 9 at the branch connector 8. The syringe connector 13 is a commercial and internationally standardised Luer connector, to which a syringe (not shown) is connected, which serves as a “pump” for the suction pump arrangement 10. At the secretion bag connector 14, also via a Luer connector, there is connected a collecting bag (not shown) for the suctioned secretion, or a different secretion container. Moreover, the secretion bag connector 14 exhibits also a check valve 15 that prevents secretion from the secretion bag being sucked back into the catheter arrangement and thus makes a pumping action possible.

This arrangement allows the physician to pump off an arbitrary quantity of fluid without having to change over a valve. First, the empty syringe attached at the syringe connector 13 is drawn back, and suctions the secretion via the catheter shaft 2, the branch connector 8, the branching hose 9 and the suction valve 11. The check valve 15 is closed at the time, such that the syringe cannot suction fluid from the secretion bag. As soon as the syringe is sufficiently filled, the physician presses the suctioned secretion again out of the syringe, at which point the suction valve 11 closes such that no secretion can be pressed back into the body cavity. Instead, the check valve 15 of the secretion bag connector 14 now opens, and the pumped-off fluid thus reaches the collecting container. This pumping stroke can be repeated as often as is required, where controlling the pumped-off quantity can be don either by summing each suctioned-off syringe content, or through an external measuring system for the bag's content. Optionally, the pumping action can be effected not through a syringe, but for example through a pumping balloon, where on grounds of hygiene a disposable syringe is to be preferred.

Once the desired quantity of fluid has been pumped off, the system can be removed like a conventional catheter. In some cases, however, it is necessary and/or medically sensible to introduce a medication into the body cavity before the catheter is removed. This too can be performed with the catheter according to the invention, without the suction pump arrangement 10 first having to be removed. In order to prevent an agent introduced via the inlet 5 not reaching the body cavity via the catheter shaft 2, but instead flowing via the branching hose 9, the suction valve 11 and the check valve 15 to the secretion bag, it is sufficient to close off branching hose 9 by means of a simple hose clamp. An especially simple design of such a hose clamp 19 is shown in FIG. 4.

Sealing off the branching hose 9 is also necessary, if between individual suction procedures an irrigating liquid needs to be introduced into the pleural cavity, for example in order to loosen consolidated substances before suctioning off. In both aforementioned cases the medication or the irrigating liquid can be introduced either via a syringe needle, which is inserted into needle inlet 5, or via another feed inlet that can be connected tightly via a Luer connector at the proximal end 4 of the catheter 3.

FIG. 5 shows an alternative embodiment, in which instead of the check valve 15 shown in FIG. 1, a bill or lip valve is arranged in the Luer lock of the secretion bag connector 14. The functional principle is analogous to the one described above, where the advantages of the cost-effective bill valve are also demonstrated in the secretion bag connector 14.

As a result of the high flexibility which the use of lip valves offers, the device according to the invention can be used to suction off both liquids and gases from the body cavity.

A further embodiment of suction pump unit 10 of the pleural puncture catheter according to the invention is shown in FIG. 6. The entire suction pump unit 10 is integrated in a single very compact component and designed as a T-piece. The T-piece is shown in part-section in FIG. 6, in order to show the valves and the course of the lumen, the connectors and the valves.

In this embodiment, membrane check valves come into use which, due to their small installation dimensions, make possible a very compact construction. Starting from the bifurcation 12, the branch 16 to the syringe connector 13 in the one direction and the branch 17 to the secretion bag connector 14 in the other direction form a cross-piece with an essentially straight traversing lumen, which is only interrupted by the check valve 15. The check valve 15 is arranged before the secretion bag connector 14 and allows a flow to the connector 14 of the secretion bag. At the bifurcation 12, the valve outlet 25 of the suction valve 11 opens into the cross-piece.

The structure of the membrane check valves is shown by way of example in FIGS. 7 to 9. FIG. 7 shows a cross-sectional view of the valve in a closed position. The closing of the valve is effected by a disc-shaped valve membrane 22, which is arranged inside a valve chamber 26 such that the membrane 22 at its central section covers the valve entrance 24 that opens into the valve chamber. The valve membrane 22 can be firmly connected at its margin with the valve housing, for example through gluing or clamping, as indicated in FIG. 7, however it could also be inserted into the valve chamber 26 as floatingly mobile. When the pressure in the valve outlet 25 rises, the valve membrane 22 is pushed against the mouth of valve inlet 24 and the flow is thus blocked. Conversely in the case of rising pressure in the flow direction, the membrane is lifted like a bubble from the mouth of valve inlet 24. In the radial periphery of the valve membrane 22 there are arranged membrane openings 23, through which with a lifted membrane the medium flows and reaches the valve outlet 25. This opened valve position is shown schematically in FIG. 8, FIG. 9 shows the valve membrane 22 in a plan view, where the radial arrangement of the membrane openings 23 is recognisable. Instead of the valve membrane 22, a rigid disc could also be used that is arranged floating in the valve chamber 26, such that it is pressed against the mouth of valve inlet 24 on locking and lifts from it on opening. When designing the valve chamber and the membrane or the disc, it needs to be ensured that the locking disc or membrane cannot cover up the valve outlet in the open valve position.

FIG. 10 shows the pleural puncture catheter with the suction pump arrangement 10 designed as a T-piece. If low pressure is created in the T-piece via a syringe placed at the connector 13, the medium to be suctioned out of the body cavity via the catheter is suctioned via the line 9, the suction valve 11 and the branch 16 of the T-piece into the syringe. The check valve 15 prevents medium already located in the secretion bag from penetrating back into the suction pump unit 10. Afterwards, pressure on the medium located in the syringe is exerted by the syringe, such that it is dislodged from the syringe into the cross-piece of the T-piece and via the check valve 15 into the secretion bag attached at the connector 14. In this situation the suction valve 11 prevents the medium from getting back into the catheter and furthermore where applicable also additionally into the body cavity.

1. Medical device in the form of a catheter (3) for supplying and/or removing fluid into and/or from, especially body cavities, especially the pleural cavity, the device exhibiting a catheter head (1) and a catheter shaft (2), where the catheter head (1) exhibits at the proximal end (4) an inlet (5) provided with one or several sealing and/or stop valves (6, 7) for inserting a hollow or Verres needle (18) into the catheter (3), and where in the area between the sealing and/or stop valve(s) and the section of the catheter shaft (2) designed for insertion into the body there is provided a branch connection (8), which is connected preferably via a branching hose (9) with a suction pump arrangement (10), characterised in that the branching hose (9) leads to a suction valve (11) which allows the flow of a fluid in the direction of the suction device, but blocks the flow in the opposite direction, where the outlet of the suction valve (11) opens into a bifurcation (12), from which one branch (16) leads to a suction device connector preferably designed as a Luer lock, especially a syringe connector (13), and whose other branch (17) leads to a secretion bag connector (14), where between this branch (17) and the secretion bag connector (14) there is arranged a check valve (15) that blocks the backflow from the secretion bag.

2. Medical device according to claim 1, characterised in that the secretion bag connector (14) is designed as a Luer lock, where the check valve (15) that prevents the backflow from the secretion bag is a bill or lip valve or a membrane check valve, which is arranged on the inside of the Luer lock.

3. Medical device according to claim 1 or 2, characterised in that the suction valve (11) is designed as a bill or lip valve or as a membrane check valve.

4. Medical device according to one of the aforementioned claims, characterised in that the branching hose (9) can be closed off between the catheter (3) and the suction valve (11) preferably by means of a hose clamp (19).

5. Medical device according to one of the aforementioned claims, characterised in that the needle inlet (5) for introducing the hollow or Verres needle (18) exhibits an inwardly directed bill or lip valve designed as a push-through valve (6), which abuts and seals at the perimeter of the inserted hollow or Verres needle (18), and with the needle withdrawn blocks in both directions the fluid passage under the pressure differences that usually occur between the body cavity and the outside.

6. Medical device according to claim 5, characterised in that a preferably manually operated stop valve (7) is arranged between the push-through valve (6) and the branching connector (8).

7. Medical device according to claim 1, characterised in that the suction pump arrangement (10), including suction valve (11), bifurcation (12), the branch (16) to the syringe connector (13), the branch (17) to the secretion bag connector (14), the syringe connector (13), the secretion bag connector (14) and the check valve (15) are integrated in one common component.

8. Medical device according to claim 7, characterised in that the suction pump arrangement (10) is designed as a T-piece, where the suction valve (11) and/or the check valve (15) are/is designed as a membrane check valve.
